(19) United States
(12) Patent Application Publication
(10) Pub. No.: US 2022/0160994 A1
(43) Pub. Date: May 26, 2022

(54) NON-EEG APPARATUS, SYSTEM, AND METHOD FOR SLOW-WAVE SLEEP MODULATION THERAPY FOR TREATMENT, PREVENTION, AND/OR MITIGATION OF INSOMNIA

(71) Applicant: Click Therapeutics, Inc., New York, NY (US)

(72) Inventor: Anthony Martorell, New York, NY (US)

(73) Assignee: Click Therapeutics, Inc., New York, NY (US)

(21) Appl. No.: 17/286,034

(22) PCT Filed: Sep. 3, 2020
(86) PCT No.: PCT/US2020/049137

§ 371 Date: Apr. 16, 2021

Related U.S. Application Data
(60) Provisional application No. 62/895,439, filed on Sep. 3, 2019.

(51) Int. Cl. A61M 21/02

(52) U.S. Cl. CPC A61M 21/02; A61M 2021/0022

(57) ABSTRACT

A computer system for slow-wave sleep modulation therapy comprising one or more processors, one or more computer-readable memories, and one or more computer-readable storage devices, and program instructions stored on at least one of the one or more storage devices for execution by at least one of the one or more processors via at least one of the one or more memories is provided. The system includes determining a need for sleep wave modulation, wherein the determining the need for sleep wave modulation is not by electroencephalography (“EEG”), and providing the modulation via slow-wave stimulus to a user, wherein the slow-wave stimulus comprises oscillations of a frequency between 0.25 and 8 Hz, and wherein, in response to the slow-wave stimulus, a brain frequency of the user receiving the slow-wave stimulus is modulated closer to the frequency of the slow-wave stimulus than the brain frequency prior to receiving the slow-wave stimulus.

### PRIORITY

The present application claims priority to U.S. Provisional Patent Application No. 62/895,439, which was filed in the United States Patent and Trademark Office on Sep. 3, 2019, the entire disclosure of which is incorporated herein by reference.

### INTRODUCTION

Embodiments of the invention relate generally to treatment of insomnia disorders, and more particularly, to methods of treatment of insomnia using slow-wave modulation therapies.

Global epidemiological estimates of insomnia symptoms are approximately 30-35%, while the average prevalence rate of insomnia disorder is 10% for studies that used the Diagnostic and Statistical Manual of Mental Disorders IV (DSM IV) criteria. Insomnia is characterized by nocturnal and diurnal symptoms, including daytime tiredness, irritability, and predominantly involves a dissatisfaction with sleep quality or duration. Clinical studies indicate that insomnia symptoms may be attributed to the inability of the brain to switch from aroused to sleep-states. Accumulating electroencephalography (EEG) evidence suggests that insomnia characteristics may be attributed to atypical high frequency neural activity at sleep onset, and during both non-rapid eye movement (NREM) and rapid eye movement (REM) sleep. Specifically, insomnia patients have shown increased beta wave (14-35 Hertz (Hz)) activity during sleep states in place of desired slow-wave alpha (8-12 Hz) and delta (1-4 Hz) oscillations, which should be predominant.

Polysomnography (PSG) recordings show that patients with insomnia exhibit an abnormal amount of beta wave (14-35 Hz) activity during sleep onset and/or NREM sleep when compared to those without insomnia symptoms. Elevated forms of beta activity during sleep is considered potentially pathological, as beta oscillations are functionally associated with arousal states, such as attention, perception, and cognitive function in mammals. Lower frequency oscillation states in the alpha and delta ranges are functionally associated with sleep-states (REM/NREM) and/or memory consolidation, a key role of sleep. Thus, the occurrence of beta activity during sleep-states in insomnia patients may provide a physiological explanation for the disposition to frequent sleep-awakenings and difficulty of sleep onset, potentially due to the brain being inappropriately primed for sensory processing and cognitive function.

Current pharmacological therapies such as sleep-medications provide acute periods of sleep relief. However they are incapable of providing reliable long-term sleep solutions to insomnia. Specifically, sleep-medications do not provide the induction of natural sleep cycles. Moreover, such pharmacological therapies often result in dependence, and may produce a host of side effects.

Recent evidence suggests non-invasive auditory and visual stimulations may be useful for modulating neuronal oscillations. Neuronal oscillations may be brainwaves that are rhythmic or repetitive patterns of neural activity, typically in the central nervous system. In such instances, modulation of neural activity using sensory stimulation has been indicated to directly affect cognitive function and behavior, and has been recently investigated for its potential to affect slow-wave sleep states.

It would be desirable, therefore, to provide apparatuses, systems and methods for identifying specific stimuli and frequencies for modulating neural activity.

It would be additionally desirable to provide apparatuses, systems and methods for affecting slow-wave sleep states.

It would be further desirable to provide a non-invasive sensory stimulation system that can modulate slow-wave sleep oscillations.

It would be yet further desirable to provide systems and methods that can monitor physiological indicators during sleep, such as heart rate (pulse), blood pressure, body temperature, body movement patterns, mobility, respiration rate, and other suitable indicators.

It would be yet further desirable to provide an apparatus, system and method for modulating non-invasive sensory stimuli for the purpose of slow-wave oscillation induction using body physiological indicators.

It would be further desirable to provide apparatuses, systems and methods for correcting and increasing accuracy of monitoring and processing the physiological indicators based on the above.

It would be desirable, therefore, to provide apparatuses, systems and methods for utilizing an improved non-invasive sensory stimulation system for modulating slow-wave sleep states.

### BRIEF DESCRIPTION OF THE DRAWINGS

FIG. 1 illustrates a block diagram of a distributed computer system that can implement one or more aspects of an embodiment of the present invention;

FIG. 2 illustrates a block diagram of an electronic device that can implement one or more aspects of an embodiment of the invention; and

FIG. 3 illustrates a slow-wave system overview (closed loop) according to one or more aspects of an embodiment of the present invention;

FIGS. 4A-4H show source code that can implement one or more aspects of an embodiment of the present invention.

While the invention is described with reference to the above drawings, the drawings are intended to be illustrative, and the invention contemplates other embodiments within the spirit of the invention.

### DETAILED DESCRIPTION

The present invention will now be described more fully hereinafter with reference to the accompanying drawings which show, by way of illustration, specific embodiments by which the invention may be practiced. This invention may, however, be embodied in many different forms and should not be construed as limited to the embodiments set forth herein; rather, these embodiments are provided so that this disclosure will be thorough and complete, and will fully convey the scope of the invention to those skilled in the art. Among other things, the present invention may be embodied as devices or methods. Accordingly, the present invention may take the form of an entirely hardware embodiment, an entirely software embodiment, or an embodiment combining software and hardware aspects. The following detailed description is, therefore, not to be taken in a limiting sense.

Throughout the specification and claims, the following terms take the meanings explicitly associated herein, unless the context clearly dictates otherwise. The phrases “in one embodiment,” “in an embodiment,” and the like, as used herein, does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” includes plural references. The meaning of “in” includes “in” and “on.”

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

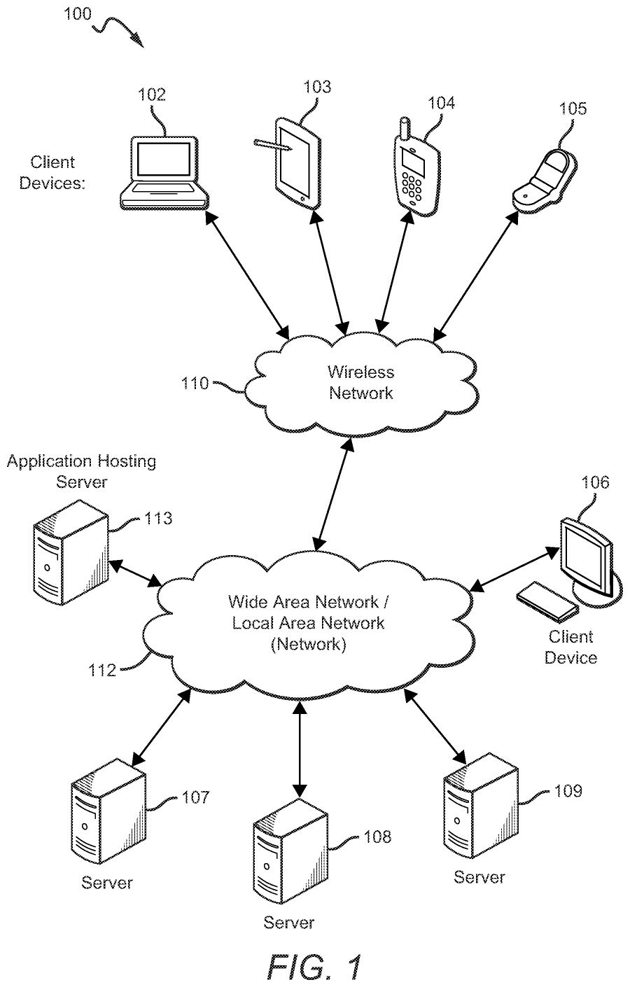

FIG. 1 illustrates components of one embodiment of an environment in which the invention may be practiced. Not all of the components may be required to practice the invention, and variations in the arrangement and type of the components may be made without departing from the spirit or scope of the invention. As shown, the system 100 includes one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. Various of the client devices 102-106 may include, for example, desktop computers, laptop computers, set top boxes, tablets, cell phones, smart phones, smart speakers, wearable devices (such as the Apple Watch) and the like. The servers 107-109 can include, for example, one or more application servers, content servers, search servers, and the like. FIG. 1 also illustrates application hosting server 113.

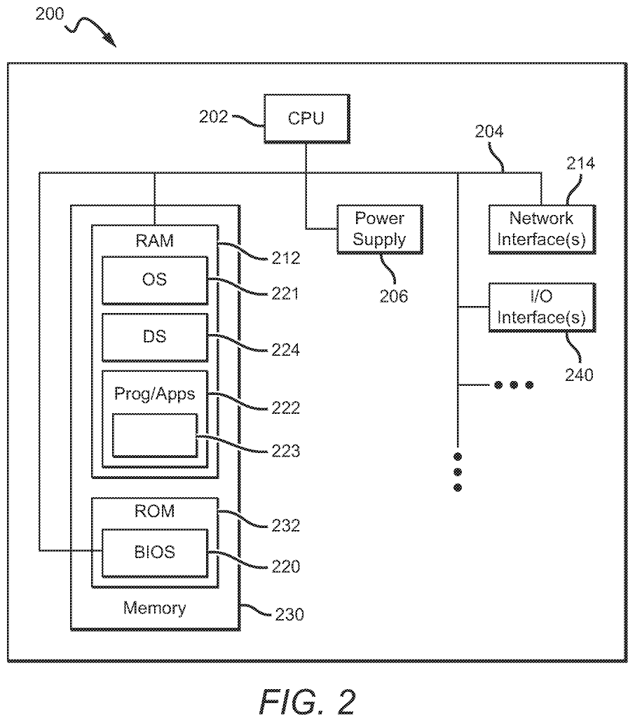

FIG. 2 illustrates a block diagram of an electronic device 200 that can implement one or more aspects of systems and methods for slow-wave sleep modulation therapy according to one embodiment of the invention. Instances of the electronic device 200 may include servers, e.g., servers 107-109, and client devices, e.g., client devices 102-106. In general, the electronic device 200 can include a processor/CPU 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 200. A touchscreen may determine whether a user is providing input by, for example, determining whether the user is touching the touchscreen with a part of the user's body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the slow-wave sleep modulation therapy program 223. The ROM 232 can also include Basic Input/Output System (BIOS) 220 of the electronic device.

Software aspects of the slow-wave sleep modulation therapy program 223 are intended to broadly include or represent all programming, applications, algorithms, models, software and other tools necessary to implement or facilitate methods and systems according to embodiments of the invention. The elements may exist on a single computer or be distributed among multiple computers, servers, devices or entities.

The power supply 206 contains one or more power components, and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can ease processing performed by the processor 202.

Where the electronic device 200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the slow-wave sleep modulation therapy for treatment, prevention, and/or mitigation of insomnia, via a network to another device. Also, an application server may, for example, host a web site that can provide a user interface for administration of example aspects of the apparatus, system and method for slow-wave sleep modulation therapy for treatment, prevention, and/or mitigation of insomnia.

Any computing device capable of sending, receiving, and processing data over a wired and/or a wireless network may act as a server, such as in facilitating aspects of implementations of the apparatus, system and method for slow-wave sleep modulation therapy for treatment, prevention, and/or mitigation of insomnia. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method for slow-wave sleep modulation therapy for treatment, prevention, and/or mitigation of insomnia. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method for slow-wave sleep modulation therapy for treatment, prevention, and/or mitigation of insomnia. Content may include, for example, text, images, audio, video, and the like.

In example aspects of the apparatus, system and method for slow-wave sleep modulation therapy for treatment, prevention, and/or mitigation of insomnia, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method for slow-wave sleep modulation therapy for treatment, prevention, and/or mitigation of insomnia, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method for slow-wave sleep modulation therapy for treatment, prevention, and/or mitigation of insomnia, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games (such as fantasy sports leagues), receiving advertising, watching locally stored or streamed video, or participating in social networks.

In example aspects of the apparatus, system and method for slow-wave sleep modulation therapy for treatment, prevention, and/or mitigation of insomnia, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method for slow-wave sleep modulation therapy for treatment, prevention, and/or mitigation of insomnia, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method for slow-wave sleep modulation therapy for treatment, prevention, and/or mitigation of insomnia, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present invention include apparatuses, systems, and methods for slow-wave sleep modulation therapy for the treatment, prevention, and/or mitigation of insomnia. Embodiments of the present invention may be implemented on one or more of client devices 102-106, which are communicatively coupled to servers including servers 107-109. Moreover, client devices 102-106 may be communicatively (wirelessly or wired) coupled to one another. In particular, software aspects of the above may be implemented in slow-wave sleep modulation therapy program 223. The slow-wave sleep modulation therapy program 223 may be implemented on one or more client devices 102-106, one or more servers 107-109, and 113, or a combination of one or more client devices 102-106, and one or more servers 107-109 and 113.

Insomnia is characterized by disrupted sleep patterns, which can manifest as difficulty falling asleep or difficulty remaining asleep. Insomnia may be classified broadly as either cognitive or physiological-based insomnia.

In certain instances of physiological-based insomnia, dysregulation of circadian rhythms may be a cause of sleep difficulty. The dysregulation may result from a variety of reasons, including mutations in clock genes responsible for the regulation of the awake-sleep cycle, as well as a shifted circadian pacemaker, where the insomnia subject naturally falls asleep much earlier or later than normal and thus wakes up at inappropriate times for a typical day-time schedule. Over time, this dysregulation will result in tiredness and “sleep debt” during the daytime. Alternatively, the dysregulation may result in abnormal levels of beta activity during sleep periods. For example, 12-30 Hz beta activity during sleep periods may correlate with periods of hyperarousal, where the insomnia subject cannot fall asleep, and/or is easily awoken.

In an embodiment, apparatuses, systems, and methods for slow-wave sleep modulation therapy for treatment, prevention, and/or mitigation of insomnia (hereinafter, collectively referred to as “the therapy”) in an individual are provided, which may be implemented at least in part in software aspects of the slow-wave sleep modulation therapy program 223.

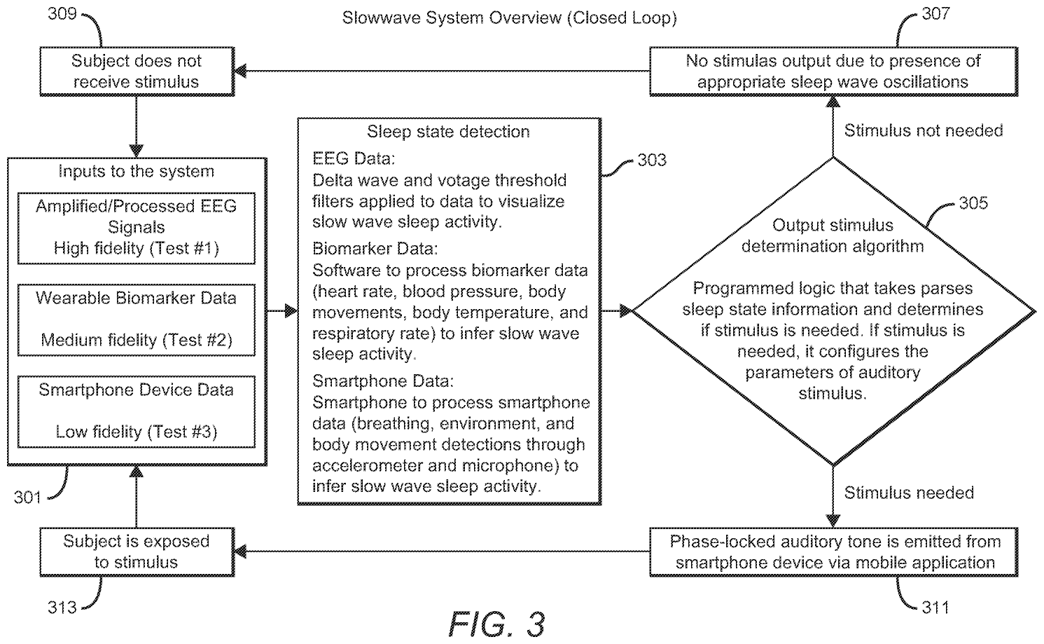

The therapy may be discussed with respect to FIG. 3, which illustrates a slow-wave system overview (closed loop), according to one or more aspects of an embodiment of the present invention. In step 301, inputs are received from the user, which may include any combination of EEG signals, wearable biomarker data (for example, from a smart phone or smart watch), and/or other smart phone device data. In step 303, sleep state detection occurs based on the data generated in step 301. In step 305, the output stimulus determination algorithm determines whether a stimulus is required or not. If it is determined in step 305 that a stimulus is not required, the process proceed to step 307, where no stimulus is output. In step 309, the subject then does not receive any stimulus and the system returns to the initial state 301. On the other hand, if at step 305, the output stimulus determination algorithm, running on the slow-wave sleep modulation therapy program 223, determines that a stimulus is required, it proceeds to step 311, where, for example, a phase-locked auditory tone is emitted by an output device, such as a smartphone (through speakers or earphones, for example). At step 313, the subject (user listening to the speakers or earphones) is then exposed to the stimulus. The slow-wave sleep modulation therapy program 223 then returns to initial state 301. Further aspects of the embodiments of the present invention are discussed below.

The therapy may utilize non-invasive sensory stimuli therapy. Although the therapy may produce a closed-loop slow-wave auditory sensory stimulation using electroencephalography (“EEG”) to determine whether to output a stimulus in step 305, many users may not have access to an EEG apparatus. For example, a client device 102-106 may be a smartphone and not include EEG electrodes and the like. However, the smartphone may include various sensors that may be used to determine a need for sleep wave modulation in step 305 without using EEG. The therapy may produce a closed-loop slow-wave auditory sensory stimulation with various physiological measures, such as heart rate variability (HRV), average heart rate (beats per minute, BPM), maximum rate of decline (MROD) in body temperature, average body temperature (° F.), average blood pressure (mmHg), and muscle activity, for the entrainment of slow-wave oscillations in real-time during sleep. Specifically, in accordance with an embodiment, the therapy utilizes auditory stimuli of 50 ms long bursts of pink 1/f noise, emitted as square-waves, and calibrated between 30-50 dB Sound Pressure Level (SPL). These tones would occur at a slow-wave frequency of approximately 4 Hz (4 tones per second with an inter-stimulus interval of 200 ms). Auditory tone intensity (dB) may be individually adjusted per subject, starting from 30 dB in 5 dB increments until the subject is comfortable at the loudest intensity (50 dB being the maximum). The therapy is designed to cause the individual to be: (1) induced with slow-wave oscillations; (2) modulate real-time slow-wave oscillations based on sleep-state dependent physiological indicators; and/or (3) indirectly modulate other frequency-band oscillations (e.g. beta oscillations). “Oscillation” refers to a rhythmic pattern of neural activity in the central nervous system. “Slow-wave” refers to periods of sleep where slow-frequency oscillations ranging from alpha to delta (0.25-8 Hz) predominantly occur, with a similar frequency to the non-invasive sensory stimuli. Thus, as disclosed herein, embodiments of the invention may include determining a need for slow wave modulation, and providing the modulation via slow-wave stimulus.

In one embodiment, sleep modulation may occur via the modulation of slow frequency sensory stimulation. The therapy may include administration of a stimulus, or plurality of stimuli. The stimulus may be any suitable stimulus, including, but not limited to auditory, visual, mechanical, electrical, and haptic. In one embodiment, an auditory stimulus may include 50 ms long bursts of pink 1/f noise, emitted as square-waves, and calibrated between 30-50 dB SPL. In an embodiment, the stimulus/stimuli may be administered at different frequencies. A frequency may be chosen for a specific stimulus, in order to trigger and/or modulate a desired brain frequency and/or synchronize with the detected slow-wave frequency. For example, an auditory tone stimulus at approximately 4 Hz can be used in order to induce and/or modulate 4 Hz neural oscillations. In the case an auditory stimulus is used, an electroacoustic transducer may be used to convert an electrical audio signal into a corresponding sound stimulus. For example, the sound stimulus may be a click-train with the desired frequency (Hz). The click-train may be a sound stimulus that is triggered mechanically or electrically (such as via a clicker), or via a closed-loop fashion where the sound occurs in response to specific physiological marker(s). The sound stimulus may alternatively, or in addition, be long tones and not clicks. In a further embodiment, the stimulus/stimuli may be administered for a specified period of time, such as for a period of seconds (such as approximately 20 seconds, which may be 19-21 seconds), minutes or even hours, or following specific physiological indicators during sleep, such as increased HRV. Stimulation following specific physiological indicators during sleep are administered for as long as the indicators occur during sleep periods. For example, a sleep period with increased HRV would induce slow-wave auditory stimulation for as long as the HRV is above baseline.

Other key factors in the physiology of sleep affected in insomnia are the autonomic regulation of body temperature and cardiovascular functions before and during sleep onset, and the transitions to different sleep stages. The maximum rate of decline (MROD) in body temperature, or the curve to the lowest point in body temperature, is a consistent physiological indicator of sleep onset in an individual, and may be used to approximate the most user-appropriate time to fall asleep at night. In insomnia subjects, MROD in body temperature is often shifted earlier or later in the day, thus contributing to difficulties in sleep onset. Thus, sleep hygiene methods such as taking a hot bath 2-hours before bed can be performed to induce MROD in body temperature. Average body temperature (° F.) is another physiological indicator of sleep, where body temperature follows a unimodal curve during sleep, peaking at the lowest temperature at midnight and gradually increasing in temperature approaching dawn. Another sleep cardiovascular physiological measure is heart rate variability (HRV). HRV is the time interval between heartbeats over time and is strongly affected by sleep stage. In normal sleepers, during the transition from wake to NREM (which consists mainly of slow-wave oscillations) sleep, there is an increase in high (0.015-0.04 Hz) frequency and a decrease in low frequency (0.04-0.015 Hz) HRV. During NREM to REM sleep there is a reduction in total HRV. Insomnia subjects have showed opposite HRV measures during sleep, specifically increased low-frequency and decreased high frequency HRV during sleep. Other common cardiovascular indicators of sleep include average heart rate (beats per minute) (HR) and blood pressure (mmHg²) (BP), both decreasing from awake to NREM sleep transition, and increasing during NREM to REM transitions, and may be utilized.

In one embodiment, individuals with insomnia are known to have reduced slow-wave oscillation brain activity during sleep. In one aspect, a method of reducing insomnia symptoms may include: (1) slow-wave auditory stimulation following specific physiological indicators of slow-wave sleep activity; (2) determining a personalized user stimulus through changes in sound intensity and/or tone; (3) determining specific points in time to trigger slow-wave oscillation therapy through various physiological indicators during sleep, including HRV, average heart rate, MROD in body temperature, average body temperature, and average blood pressure; (4) determining the duration of stimulation needed indicated through periods of physiological indicators; (5) providing non-invasive neural stimulation for insomnia via the stimulus; and (6) monitoring, via the various physiological indicators in real-time, the result of slow-wave sensory stimulation on sleep, such as increased NREM/REM activity.

Determining the right point in time to trigger the slow-wave oscillation therapy may include: (1) analyzing body temperature to calculate average changes over time and the MROD, so that stimulation may be triggered and/or would occur below certain temperature thresholds during sleep—temperature thresholds would initially be determined in a user-specific model where baseline temperature is calculated throughout sleep; (2) monitoring heart rate over time to measure average heart beat count per minute as well as HRV, so that stimulation may be triggered and/or would occur during specific heart rates indicated with sleep stages—user-specific heart rates would be determined during baseline monitoring; (3) monitoring blood pressure over time to determine changes in sleep stage, user-specific blood-pressure changes would be determined during baseline monitoring; (4) finger motility changes, based on the knowledge that throughout sleep stages there are periods of arousal; (5) limb movement, based on the knowledge that throughout sleep stages there are periods of arousal; and (6) based on these stimuli, an auditory stimulus at a slow-wave frequency will be induced at specific times during sleep.

In an embodiment, determining the appropriate timing and trigger of slow-wave modulation in an individual may be determined via various physiological indicators from wearable monitors and/or devices. In an embodiment, monitors or sensors capable of detecting changes in body temperature, heart rate, blood pressure, and movement would be utilized. For example, a device may be placed adjacent to digits (fingers or toes), and/or wrists. In another example, sensors may be placed on digits (fingers, toes) and/or wrists, stylized as flexible bands and/or rings. Detection of heart rate may be possible via photoplethysmography, or the usage of light-emitting-diodes (LED) which emit light and/or infrared light, while a receiver captures the absorbance of light over time to calculate heart rate. Blood pressure may be measured via a haptic device such as a ring or band that has an inflatable bladder and compresses skin for small periods of time. Limb movement detection may be performed through a device that includes motion-detector sensors that are sensitive to minute changes in movement, such as an ultrasound detector that transmits and receives its own ultrasound waves continuously, and thus any break in its detection indicates movement. Thus, detection of sleep states may occur via continuous physiological monitoring, and an auditory tone would then be emitted in real-time to modulate and/or synchronize with a concurrent sleep phase.

In a further embodiment, the determining of the right point in time to trigger slow-wave oscillation therapy may occur after a delay period, or a predetermined period of time, following an EEG signal or physiological indicator, such as the maximum rate of decline body temperature. The maximum rate of decline in body temperature is the slope of body temperature over time that is the steepest. That is, the delay period following a maximum rate of decline in body is an indicator for when individuals may be begin to “fall asleep.” Thus, the maximum rate of decline in body temperature may be determined and/or calculated. Once that specified temperature is reached, and the delay period sets in, slow-wave oscillation therapy may be triggered.

In yet another embodiment, a physiological indicator, such as heart rate, may be used as an indicator of slow-wave onset. For example, once a specified/predetermined heart rate has been reached, slow-wave oscillation therapy may be triggered. Heart rate may be user-specific, and measured during baseline monitoring to determine the ranges of heart rates indicative of sleep states. Therefore, heart rate and/or blood pressure of the individual is monitored. For example, a pulse sensor or pulse oximeter may be used to monitor heart rate. In a further example, light pulses may be placed adjacent to the skin and used to calculate pulse rates, based on perfusion. Thus, during a nighttime period, the rate of decline in body temperature is monitored. Once the maximum rate of decline in body temperature has been reached, heart rate or blood pressure may be monitored. If it is determined that the heart rate or blood pressure has decreased below a significant user-specific baseline threshold, slow-wave oscillation may be triggered due to an indicator of sleep state. In accordance with this embodiment, a reduction in heart rate or blood pressure may be used to indicate a beginning of a first phase of sleep. Therefore, the reduction in temperature or heart rate may be used to predict onset of slow-waves, and therefore indicate a time at which to provide slow-wave sensory stimulation.

Thus, in certain embodiments, an embodiment of the invention includes inducing slow-wave oscillations by stimulating slow-wave sensory stimuli, as well as determining the proper stimulus, and proper timing for the sleep wave modulation.

In another embodiment, instead of receiving biometric inputs (such as body temperature, pulse rate, and the like), the user may manually input sleep start and sleep end time and the proper stimulus will be administered based on the information entered by the user.

In an embodiment, personalized regimens of varying duration and longevity are provided, whereby each individual's form of stimulation may be personalized, based on the baseline physiological measurements and/or calculations. For example, if it is determined that a user responds better to clicks as opposed to long tones, clicks will be utilized more in the future. The user may also manually set their preferences (e.g., clicks as opposed to long tones, or higher or lower volume). It may also be determined, for a particular user, whether the user responds better to, for example a louder stimulus or softer stimulus. This is due to variable physiological values, in this case during sleep, in humans, where each human subject may differ in subtle differences in baseline body temperature, heart rate, and blood pressure, thus requiring sensory stimuli to occur at different intensities, periods, and durations. In another embodiment, oscillations are modulated in real-time. The treatment may also be provided for a predetermined number of nights, such as 45.

In an embodiment, the system may evaluate the occurrence of wake-level-like periods of body temperature, heart rate, and blood pressure, present during sleep onset or NREM sleep. This would in turn indicate that the individual may in the process of “waking up” or re-gaining consciousness. To prevent waking up, the system may modify the sensory stimuli by increasing its amplitude strength (intensity) to potentially increase neural slow-wave modulation. It should be noted that, in accordance with embodiments of the invention, the slow-wave oscillations are non-invasive, and are specifically formed to stimulate electrophysiological activity, and synchronize neural activity, using sensory stimuli at various frequencies.

In certain embodiments, sensory stimuli are utilized at various frequencies, with oscillations modulated at various times during sleep cycles. Thus, neural activity, and specifically an individual's slow wave modulations, are potentially modulated or altered during sleep to induce or improve sleep patterns. The stimuli may be specifically modulated to a frequency similar or identical to the desired slow-wave oscillation to be produced. For example, the frequency of the stimuli may be 10 Hz, if desired to produce a 10 Hz oscillation in the individual.

In one embodiment, the stimulation may be auditory stimulation, whereby neural activity of an individual is non-invasively modulated with auditory tones and/or signals at specific frequencies to modulate cortical neural activity. For example, a delta range of 1-4 Hz may be used (for example, for sleep), or a higher range, such as a gamma range (greater than 40 Hz) may be used.

In accordance with some embodiments, it should be noted that modulation of sleep states via slow-frequency sensory stimuli may be specifically timed to begin when physiological indicators are detected. For example, a wearable temperature monitor may detect that the maximum rate of decline in body temperature has occurred, and may then begin the slow-frequency sensory stimuli following a specific delay period and other physiological indicators. Non-invasive sensory stimuli may consist of using an auditory or other suitable stimulation, and would be used in conjunction with physiological indicators.

In an embodiment, non-invasive slow-wave sensory stimulation may be performed where a sensory stimulus such as an auditory tone is emitted during the beginning of a sleep state. That is, as disclosed above, slow-wave sleep modulation may be specifically timed to occur at a phase of a predicted or ongoing, but improper or insufficient, sleep rhythm. This sensory stimulation may potentially increase the likelihood of proper sleep induction, and elimination or reduction of insomnia.

Accordingly, novel methods for modulation of slow-wave oscillations may utilize non-invasive sensory stimuli in response to physiological indicators. The methods may include auditory stimulation feedback. For example, the auditory stimulation may include audio tones, to be delivered via speakers or headphone-like devices and is based on physiological indicators from wearable monitors. In a further example, the system may monitor for the decrease in heart rate and blood pressure using a wearable device. Once an indicator is detected, the system initiates a sensory stimulation, such as auditory stimulation, to produce and effect slow-wave oscillations in an individual.

For example, in an auditory tone stimulation, an electroacoustic transducer will convert an electrical audio signal into a corresponding sound stimulus, and the sound stimulus would include a click train with a desired frequency. Slow-frequency tones for auditory stimuli for the purpose of modulating slow-wave oscillations may be formed of bursts of pink 1/f noise of 50 ms in duration, emitted as square-waves, and calibrated between 30-50 decibels (dB) of SPL. Tones may be presented binaurally via ear headphones.

A gradient noise, instead of a monotonous tone for 50 ms in duration may also be used. Specifically, the ‘gradient noise’ may be the same pink 1/f noise distributed in a uniform curve, peaking with frequency intensity at the mid-point of the curve. This gradient noise gives the illusion of a smoother, more natural wave-like sound, while still offering a slow-frequency tone.

In a further embodiment, the stimulation may cause the reduction of increases in body temperature, heart rate, and/or blood pressure during non-REM sleep in an individual, thereby potentially effecting sleep duration and quality and reducing insomnia.

In accordance with an embodiment, the slow-wave sensory stimulation as disclosed herein may be specifically modified to address these needs. In particular, the slow-wave sensory stimulation may be modified to physiological indicators while being wireless and non-invasive.

In an embodiment, a wearable monitor measuring physiological indicator(s) is configured to transport and/or convert thermal, haptic, or other forms of a signal to a digital signal. This process may occur by means of amplification, transformation, filtering, and/or conversion using digital hardware and/or software. The digital signal is then stored electronically and analyzed. The digital signal may be analyzed by the wearable monitor, a device communicatively coupled thereto, the smart phone, and/or a server to which the signal is transmitted via a transceiver.

A wearable monitor may non-invasively capture physiological information in a manner that is quick and relatively affordable. This wearable monitor therefore allows for analysis of physiological body activity in a relatively short period of time. Moreover, the wearable monitor is specifically programmed to reduce and address inaccuracies in non-invasive insomnia monitoring systems.

Analysis of physiological indicators into digital parametrics may be performed through fast Fourier transforms and/or autoregressive algorithms, in which spectral components are identified independently of preselected frequencies.

An illustrative process may include, at a time determined to be appropriate for sleep (for example, such as between 10 PM-6 AM): (1) providing a portable, non-invasive wearable device; (2) providing a stimulus receiver (such as headphones for placement over the ear(s), any suitable form of haptic feedback receiver, a visual feedback receiver, or any other suitable device); (3) monitoring and recording physiological body activity using the non-invasive wearable device; (4) converting the monitored signal to a digital signal; (5) upon determining the beginning of a sleep state following a physiological indicator in the user, executing a stimulation with a sensory stimulation; (6) monitoring for feedback via physiological body monitors, including changes in duration of physiologically associated sleep states (e.g., NREM periods as indicated by HRV measures) and any changes to physiology, such as heart rate and/or blood pressure, during sleep; and (7) determining if an appropriate sleep period has been conducted.

Thus, as described above, physiological information from an individual is recorded and analyzed. For example, the system may utilize an electrode, such as a sticker pad electrode, a finger- or pulse-detector device, a pulse oximeter, light measurement or any other suitable devices, to record and analyze physiological information. Further, the headphones provide auditory stimuli during physiological monitoring, which is when a sensor records a type of energy from the skin. In an embodiment, when a physiological monitor indicates a measure suggesting proximity of a sleep state/period, the system runs the slow-wave sensory stimulation. This may cause modulation of slow-wave oscillations, resulting in better sleep.

In an embodiment, some physiological indicators may be synchronized with the occurrence of sensory stimuli. In order to detect physiological indications in real-time and present auditory stimuli in a timed manner, an algorithm may process signals issued from the wearable monitor(s). The signal will be processed and each time a filtered signal crosses a specified threshold (e.g., blood pressure, heart rate, and/or temperature), auditory tone(s) will be triggered and sent through a speaker at approximately 30-50 dB (or, 45 dB).

The algorithm may trigger the auditory tone at the beginning of, during, or after detection of certain physiological indicators. Setting the auditory tone during certain periods of physiological indicator detection allows for the accurate delivery of auditory stimuli.

It should be noted that an embodiment of the invention modulates the user's sleep period in real-time, developing a personalized sleep schedule based on the outcome. Slow frequency auditory stimuli, or other suitable stimuli, are triggered upon the detection of certain physiological indicators, which results in the potential modulation of slow-wave oscillations in the user.

In an embodiment, the slow-wave therapy is configured for use at specified times. For example, the slow-wave therapy may be used only at the beginning of the sleep cycle, to induce sleep. In a further example, slow-wave therapy may be used throughout the night (or other sleep period), at multiple stages, to enhance slow-wave oscillations that occur numerous times during a sleep session.

As discussed above, preferred frequencies of sleep activity, e.g., preferred slow-wave oscillation frequencies, may be alpha (8-12 Hz) and delta (1-4 Hz) wave ranges. When excessive beta (12-30 Hz) wave ranges are present, insomnia is found to be more prevalent.

Thus, embodiments disclosed herein are configured to non-invasively modulate brain activity by amplifying and/or modulating slow wave oscillations. The modulation is accomplished by utilizing stimuli, such as sound-based stimuli, haptic-based stimuli, or other suitable forms. Sleep is monitored and measured using wearable monitors or sensors that detect physiological indicators, which, at a predetermined proper time, wirelessly signals a slow-frequency auditory stimulus.

In an embodiment, exemplary auditory stimulation is formed of clicks or tones. In one embodiment, pink noise, formed of equal energy per octave, is used for auditory stimulation, with tones or clicks at approximately 30-60 dB. The frequency of the pink noise may be set to 1-12 Hz, and delivered through a speaker or wireless earphones.

In embodiments, auditory stimulation may be adjusted to different frequencies, which will depend and be modified, based on the individual's natural slow-wave oscillations during sleep.

The amplifier is configured to detect specific predetermined oscillations, and only based on those predetermined oscillations, synchronize stimuli, such as auditory tones, in response. This then produces or amplifies slow-wave oscillations in the user.

Several algorithms of embodiments of the present invention are illustrated in the tables below.

In the table below, algorithm logic using EEG is shown. The input category of this table is as follows: (1) EEG: EEG data (e.g., from a modified EEG headband device) is wirelessly transmitted to the slow-wave sleep modulation therapy program 223 running, for example, on a smart phone where it is processed and converted to an electrical audio signal via a digital electroacoustic transducer that then denotes a corresponding sound stimulus; and (2) Time Since Sleep Onset (Hours): this data informs what sleep stage an individual should be in in order to follow a normal sleep cycle.

| Processing of Signal/Firing Threshold | Output Parameters for Pink Noise |
|---|---|
| If the EEG signal meets a predetermined | Timing (specific points in time that the |
| condition(s) (e.g. 60 minutes after sleep onset | stimulus should be delivered) |
| and no detected slow-wave oscillations), then | Duration (seconds, minutes, hours) |
| the determination is: | Length of bursts |
| There is an absence of slow-wave | Inter-stimulus interval |
| oscillations when they should be present | Frequency (1-12 Hz) |
| The output will fire under the following | Sound level (30-60 dB) |
| parameters in the next column in order to | Sound type (clicks |
| induce slow-wave oscillations. | vs long tones) |
| If the EEG signal is within 0.25-4 Hz in the | Timing (specific points in time that the |
| predetermined condition(s), then the | stimulus should be delivered): Synchronized |
| determination is: | with detected slow-waves |
| There are slow-wave oscillations present | Duration (seconds, minutes, hours): |
| The output will fire under the following | Synchronized with detected slow-waves |
| parameters in the next column in order to | Length of bursts |
| amplify/enhance existing detected slow-wave | Inter-stimulus interval |
| oscillations. | Frequency (1-12 Hz): Synchronized with |
| | detected slow-waves |
| | Sound level (30-60 dB) |
| | Sound type (clicks vs long tones) |
| If the EEG signal is in the predetermined | Timing (specific points in time that the |
| condition(s) (e.g. 60 minutes after sleep onset | stimulus should be delivered) |
| and detected high frequency oscillations), | Duration (seconds, minutes, hours) |
| then the determination is: | Length of bursts |
| There is a predominant presence of high | Inter-stimulus interval |
| frequency oscillations (e.g. beta waves) when | Frequency (1-12 Hz) |
| there should not be (e.g. NREM stages) | Sound level (30-60 dB) |
| The output will fire under the following | Sound type (clicks vs long tones) |
| parameters in the next column in order to | |
| indirectly reduce amplitude of beta | |
| oscillations by inducing slow-wave | |
| oscillations. | |

In the table below, algorithm logic using biomarkers (derived from a wearable monitor such as a smart watch sensing pulse or body temperature) is shown. The input category of this table is as follows: (1) Wearable/Biomarker: The following biomarker data (derived from a wearable monitor) will be collected to infer sleep state throughout the night: Heart rate variability (HRV), Average heart rate, average blood pressure, body temperature, and body movement; and (2) Time Since Sleep Onset (Hours): this data informs what sleep stage an individual should be in in order to follow a normal sleep cycle.

| | Output Parameters |
|---|---|
| Processing of Signal/Firing Threshold | for Pink Noise |
| If the: Heart Rate Variability | Timing (specific points |
| (HRV) shows a | in time that the |
| predetermined condition, and/or | stimulus should be delivered) |
| Average Heart Rate shows a predetermined | Duration (seconds, |
| condition (e.g. 50-55 bpm), and/or | minutes, hours) |
| Average Blood Pressure shows a | Length of bursts |
| predetermined condition (e.g. 20% | Inter-stimulus interval |
| decrease in systolic/diastolic since | Frequency (1-12 Hz) |
| sleep onset), and/or | Sound level (30-60 dB) |
| Body Temperature (MROD) shows a | Sound type |
| predetermined condition, and/or, | (clicks vs long tones) |
| Body Movement shows a predetermined | |
| condition, and/or | |
| Then the determination is: | |
| The patient should have slow-wave | |
| oscillations induced and/or amplified | |
| The output will be triggered under the | |
| following parameters in the next column in | |
| order to induce/modulate slow-wave | |
| oscillations. | |

With respect to the above table, if there are contradicting signals among the different types of biomarker data (e.g. HRV signals slow-wave sleep but the MROD signals REM sleep), then determination will be based on the most reliable biomarker data for sleep state determination. Reliability of the biomarker data may be continuously determined., For example, the biomarker data, from most to least reliable, may be as follows: HRV, MROD, average heart rate, body movement, and average blood pressure.

In the table below, algorithm logic using an accelerometer and microphone is shown. The input category of this table is as follows: (1) smart phone Device: The following smartphone device data will be captured to infer sleep state: (a) Tri-axial accelerometer (motion sensor that tracks movement in every direction). May also use/include a gyroscope to measure orientation and rotation, and (b)—Microphone on smartphones can inform system of respiration rate, snoring, body movements, and noise and use such information to determine sleep state; (c) Sleep Journal data can inform system preemptively (not in real time) of the timing of predicted sleep states based on the individual's self-reported typical sleep and wake times (both the final wake time and sleep interruptions throughout the night); and (2) Time Since Sleep Onset (Hours): This data informs what sleep stage an individual should be in in order to follow a normal sleep cycle.

| Processing of Signal/Firing Threshold | Output Parameters for Pink Noise |
|---|---|
| If the Accelerometer and/or Microphone | Timing (specific points in time that the |
| signal indicates individual is not in a slow- | stimulus should be delivered) |
| wave sleep state when they should be, then | Duration (seconds, minutes, hours) |
| the output will fire under the following | Length of bursts |
| parameters in the next column in order to | Inter-stimulus interval |
| induce slow-wave oscillations. | Frequency (1-12 Hz) |
| | Sound level (30-60 dB) |
| | Sound type (clicks vs long tones) |
| If the Accelerometer and/or Microphone | Timing (specific points in time that the |
| signal indicates individual is in a slow-wave | stimulus should be delivered): Synchronized |
| sleep state but needs enhancement, then the | with detected slow waves |
| output will fire under the following | Duration (seconds, minutes, hours): |
| parameters in the next column in order to | Synchronized with detected slow-waves |
| amplify/enhance existing detected slow-wave | Length of bursts |
| oscillations. | Inter-stimulus interval |
| | Frequency (1-12 Hz): Synchronized with |
| | detected slow-waves |
| | Sound level (30-60 dB) |
| | Sound type (clicks vs long tones) |

With respect to the above table, if there are contradicting signals among the different types of smartphone device data (e.g., accelerometer data signals slow-wave sleep but the microphone data signals REM sleep), then determination will be based on which smartphone data type (accelerometer vs. microphone vs. sleep journal) is most reliable for sleep stage determination. The accelerometer is more reliable for sleep stage determination.

Considering the above, in one embodiment of the present invention, the EEG closed loop assesses the validity of an algorithm that modulates sleep based on determination of sleep state from EEG data and configuration of output parameters to create or amplify slow waves when needed. The algorithm is able to continuously detect sleep state throughout the sleeping period and modify output in real time based on changes to sleep state.

In another embodiment of the present invention, the non-EEG open loop system assesses the efficacy of an open loop system with predetermined system outputs using the validated algorithm and hypothesized sleep states based on sleep journal inputs/baseline information (bed time, sleep time, awakenings) and general insomnia sleep stage map; or constant slow-wave output throughout the night.

In yet another embodiment of the present invention, the non-EEG open loop system assesses the fidelity and efficacy of biomarker data (heart rate variability, average heart rate, average blood pressure, body movements, body temperature, respiration rate) as a form of real-time system inputs that determine the sleep state.

In yet another embodiment of the present invention, the non-EEG open loop system assesses the fidelity and efficacy of smart phone device data (accelerometer, microphone, light sensor) as a form of real-time system inputs that determine sleep state and sleep quality.

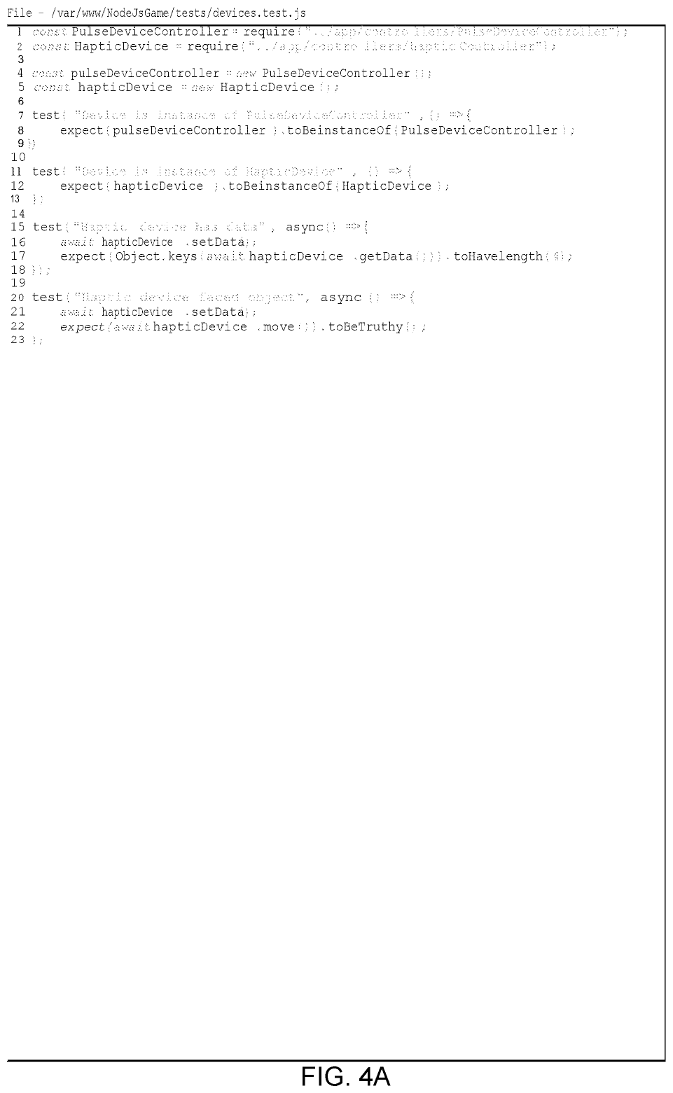

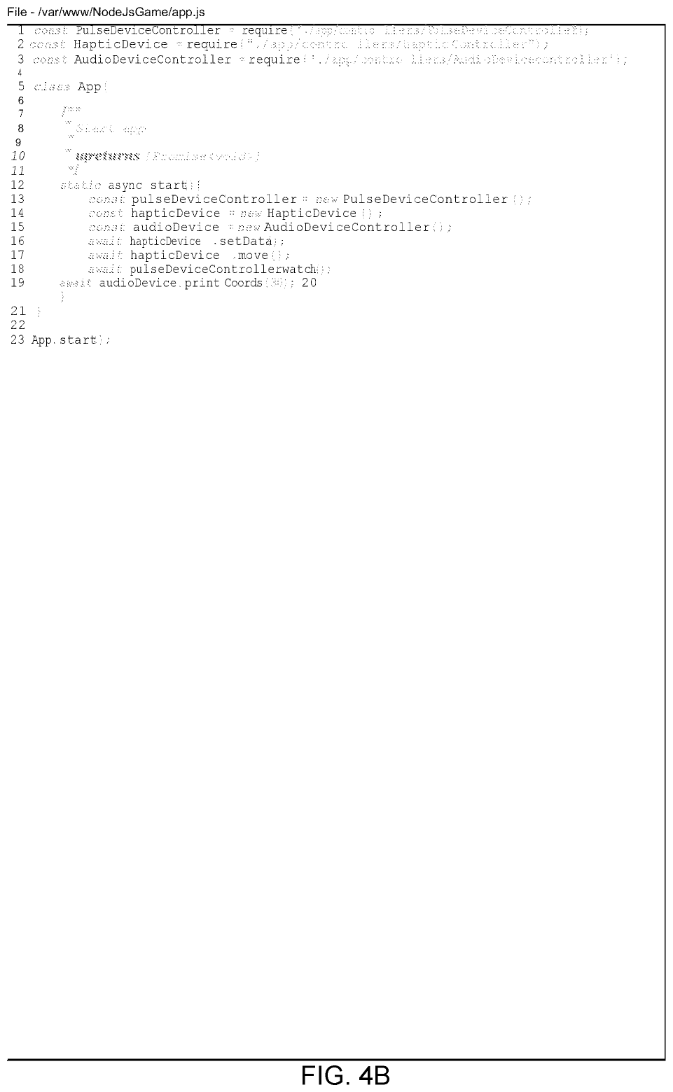

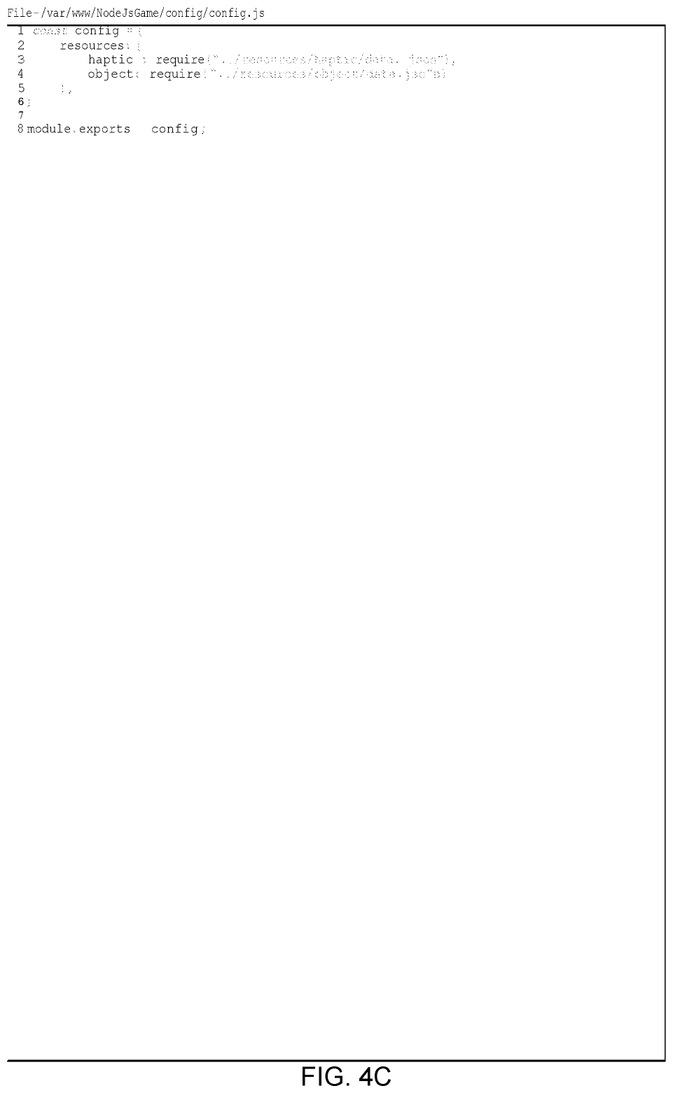

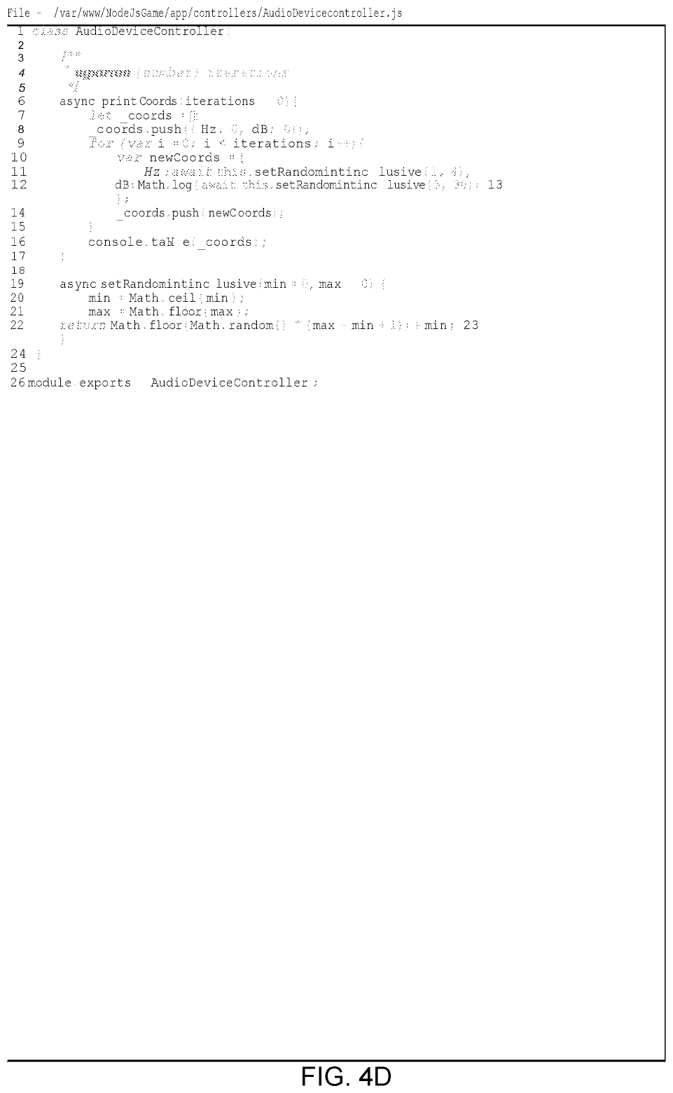

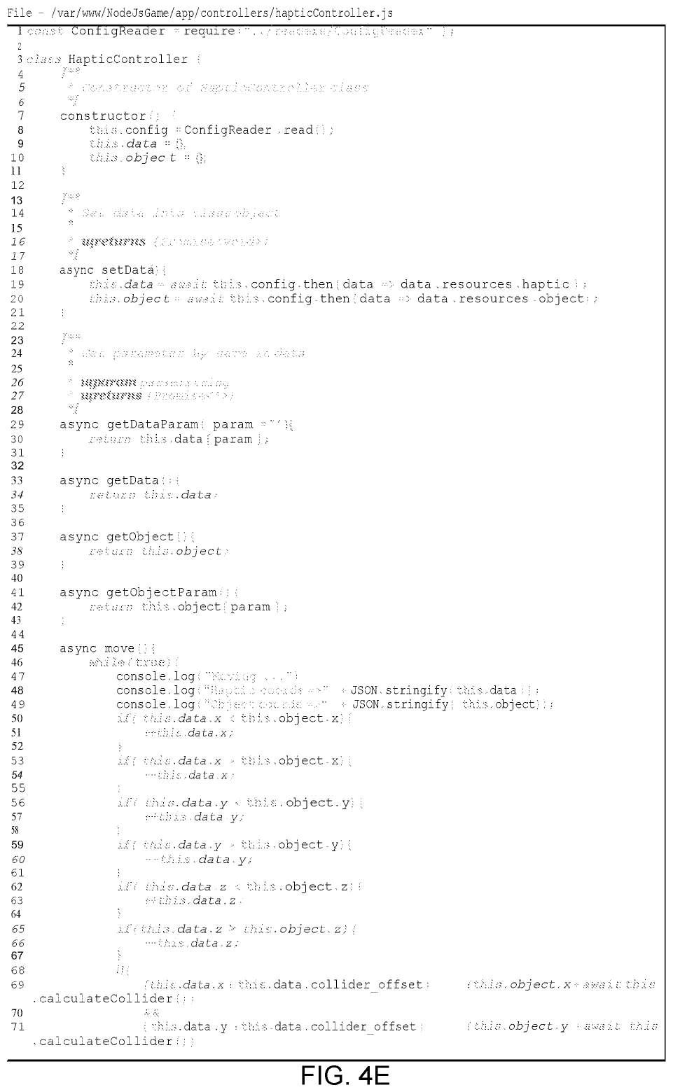

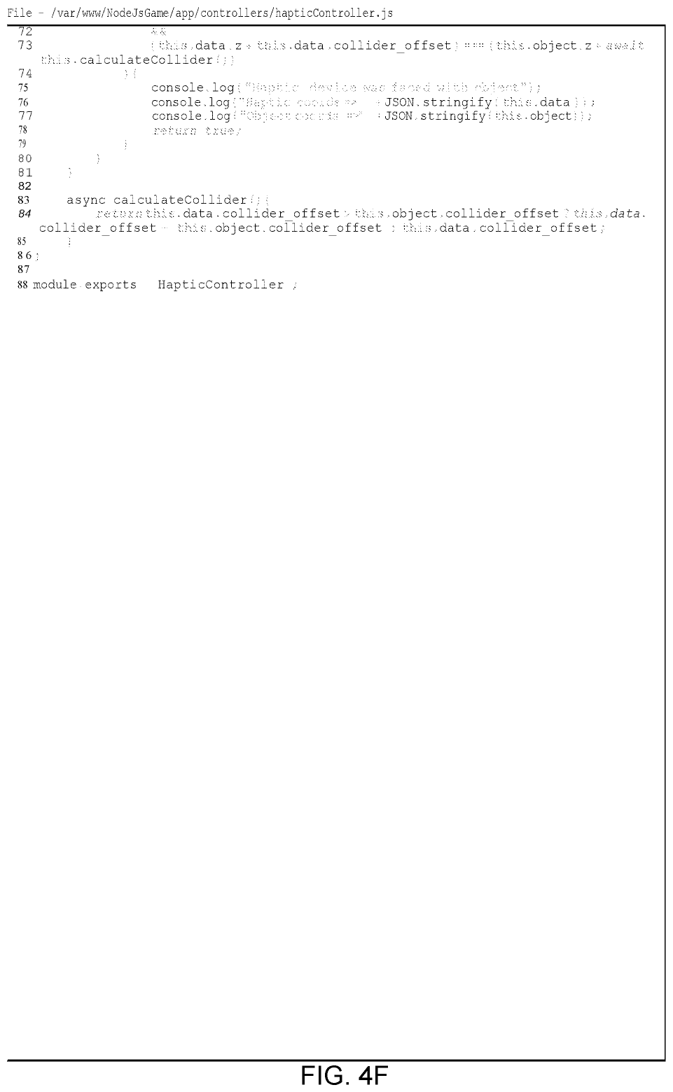

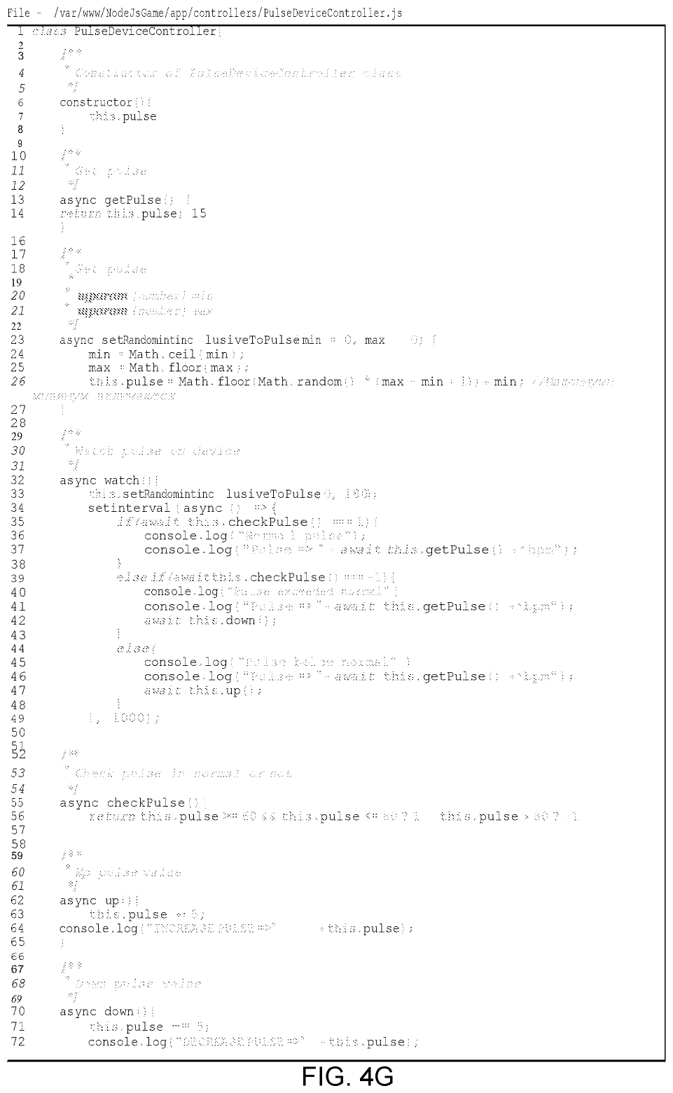

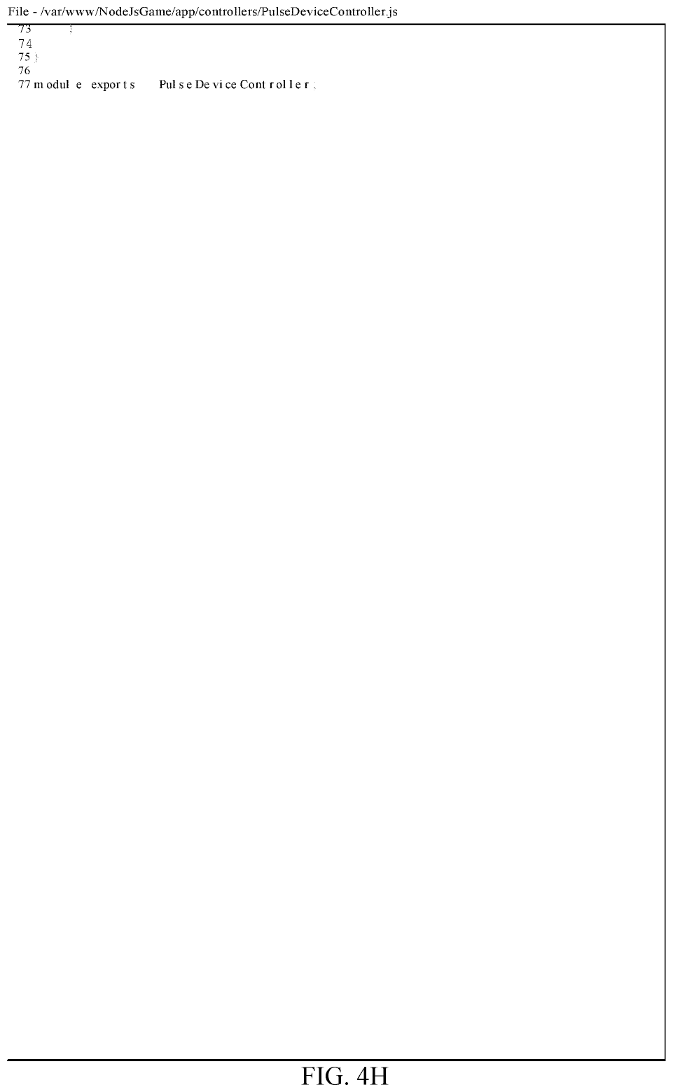

FIGS. 4A-4H, show source code that can implement one or more aspects of an embodiment of the present invention. The figures include: (1) a first algorithm configured to convert haptic force signals, obtained by a person wearing haptic sensors, to digital signals (digitalized haptic signals); (2) a second algorithm configured to compare the digitalized haptic signals with “ideal” haptic signals; and (3) a third algorithm configured to produce audio signals, particularly square waves, at approximately 4 Hz and between 30-5 dB, in proportion to the difference between the digitalized and ideal haptic signals.

In certain embodiments, the therapy may be used as part of a treatment regimen, in conjunction with the use of one or more pharmaceutical compositions. Illustrative pharmaceutical compositions known for treating insomnia that may be used in conjunction with the therapy include: (a) GABA-A receptor Agonists, including benzodiazepines and benzodiazepine receptor agonists that act on GABA receptor sites and exert sedative, anxiolytic, muscle relaxant, and/or hypnotic effects, such as Zolpidem, Zaleplon and Eszopiclone; (b) Melatonin Receptor Agonists, such as Melatonin, Ramelteon and Tasimelteon; (c) Orexin Receptor Agonists, such as Suvorexant (in doses of, for example, 5 mg, 10 mg, 15 mg, or 20 mg); (d) Histamine-1 Receptor Antagonists, such as Doxepin (in doses of, for example, 3 mg and 6 mg; (e) Selective Serotonin Reuptake Inhibitors, such as mirtazapine, fluoxetine, citalopram and sertraline; (f) Tricylic antidepressants, such as doxepin, amitriptyline, and trimipramine; and (g) other suitable anti-depressants, anti convulsants or atypical antipsychotics.

While this invention has been described in conjunction with the embodiments outlined above, many alternatives, modifications and variations will be apparent to those skilled in the art upon reading the foregoing disclosure. Accordingly, the exemplary embodiments of the invention, as set forth above, are intended to be illustrative, not limiting. Various changes may be made without departing from the spirit and scope of the invention.

What is claimed is:

1. A computer system for slow-wave sleep modulation therapy comprising one or more processors, one or more computer-readable memories, and one or more computer-readable storage devices, and program instructions stored on at least one of the one or more storage devices for execution by at least one of the one or more processors via at least one of the one or more memories, the stored program instructions comprising:
determining a need for sleep wave modulation, wherein the determining the need for sleep wave modulation is not by electroencephalography (“EEG”); and
providing the modulation via slow-wave stimulus to a user, wherein the slow-wave stimulus comprises oscillations of a frequency between 0.25 and 8 Hz, and wherein, in response to the slow-wave stimulus, a brain frequency of the user receiving the slow-wave stimulus is modulated closer to the frequency of the slow-wave stimulus than the brain frequency prior to receiving the slow-wave stimulus.

2. The slow-wave sleep modulation therapy system according to claim 1, wherein the slow-wave stimulus is at least one of an auditory stimulus, a visual stimulus, a mechanical stimulus, an electrical stimulus, and a haptic stimulus.

3. The slow-wave sleep modulation therapy system according to claim 2, wherein the slow-wave stimulus is the auditory stimulus, and wherein the auditory stimulus is delivered to a user via a speaker, and wherein an electroacoustic transducer is used to convert an electrical audio signal into the auditory stimulus.

4. The slow-wave sleep modulation therapy system according to claim 2, wherein the slow-wave stimulus is the auditory stimulus, and wherein the auditory stimulus is delivered to a user via a speaker and wherein the auditory stimulus is a click train, wherein the click train comprises a plurality of clicks.

5. The slow-wave sleep modulation therapy system according to claim 2, wherein the slow-wave stimulus is the electrical stimulus.

6. The slow-wave sleep modulation therapy system according to claim 5, wherein the electrical stimulus delivers the slow-wave stimulus by generating a plurality of clicks.

7. The slow-wave sleep modulation therapy system according to claim 5, wherein a duration of the slow-wave stimulus is approximately 20 seconds.

8. The slow-wave sleep modulation therapy system according to claim 1, wherein determining the need for sleep wave modulation comprises determining at least one of a user's heart rate, blood pressure, body movements, body temperature, or respiratory rate.

9. The slow-wave sleep modulation therapy system according to claim 8, wherein determining the need for sleep wave modulation comprises determining the respiratory rate, and the respiratory rate is determined via a microphone.

10. The slow-wave sleep modulation therapy system according to claim 8, wherein determining the need for sleep wave modulation comprises determining the body movements, and the body movements are determined via an accelerometer sensor.

11. The slow-wave sleep modulation therapy system according to claim 1, wherein the frequency of the slow-wave stimulus is 1-4 Hz.

12. The slow-wave sleep modulation therapy system according to claim 2, wherein the slow-wave stimulus is an auditory stimulus, and wherein the auditory stimulus is delivered to the user binaurally via ear headphones.

13. A computer implemented method for slow-wave sleep modulation therapy, the method comprising:
determining, by a processor, a need for sleep wave modulation, wherein the determining the need for sleep wave modulation is not by electroencephalography (“EEG”); and
providing the modulation via slow-wave stimulus to a user, wherein the slow-wave stimulus comprises oscillations of a frequency between 0.25 and 8 Hz, and wherein, in response to the slow-wave stimulus, a brain frequency of the user receiving the slow-wave stimulus is modulated closer to the frequency of the slow-wave stimulus than the brain frequency prior to receiving the slow-wave stimulus.

14. The slow-wave sleep modulation therapy method according to claim 1, wherein the slow-wave stimulus is at least one of an auditory stimulus, a visual stimulus, a mechanical stimulus, an electrical stimulus, and a haptic stimulus.

15. The slow-wave sleep modulation therapy method according to claim 16, wherein the slow-wave stimulus is the auditory stimulus, and wherein the auditory stimulus is delivered to a user via a speaker, and wherein an electroacoustic transducer is used to convert an electrical audio signal into the auditory stimulus.

16. The slow-wave sleep modulation therapy method according to claim 16, wherein the slow-wave stimulus is the auditory stimulus, and wherein the auditory stimulus is delivered to a user via a speaker and wherein the auditory stimulus is a click train, wherein the click train comprises a plurality of clicks.

17. The slow-wave sleep modulation therapy method according to claim 1, wherein determining the need for sleep wave modulation comprises determining at least one of a user's heart rate, blood pressure, body movements, body temperature, or respiratory rate.

18. The slow-wave sleep modulation therapy method according to claim 19, wherein determining the need for sleep wave modulation comprises determining the respiratory rate, and the respiratory rate is determined via a microphone.

19. The slow-wave sleep modulation therapy method according to claim 18, wherein determining the need for sleep wave modulation comprises determining the body movements, and the body movements are determined via an accelerometer sensor.
